(19) United States
(12) Patent Application Publication
(10) Pub. No.: US 2006/0286138 A1
(43) Pub. Date: Dec. 21, 2006

(54) Novel biodegradable aliphatic polyesters and pharmaceutical compositions and applications thereof

(76) Inventors: Vinod Chintamani Malshe, Maharashtra (IN); Padma Venkitachalam Devarajan, Maharashtra (IN); Sayalee Ranjan Shastri, Maharashtra (IN)

Correspondence Address: Berkeley Law and Technology Group, 1700 N. W. 167th Place, Suite 240, Beaverton, OR, 97006, US

(21) Appl. No.: 10/552,422

(22) PCT Filed: Apr. 7, 2004
(86) PCT No.: PCT/IN04/00097

§ 371 Date: Oct. 7, 2005

(30) Foreign Application Priority Data
Apr. 10, 2003 (IN) 355/MUM/2003

(51) Int. Cl. A61K 9/14; A61F 2/02; C08F 20/00

(52) U.S. Cl. 424/423; 424/486; 525/437; 977/906

(57) ABSTRACT

Novel biodegradable aliphatic polyesters derived from fatty diacids and fatty diols with even number of carbon atoms, pharmaceutical compositions and applications thereof wherein the said pharmaceutical compositions comprises at least one pharmaceutically active ingredient and the said biodegradable aliphatic polyester and the said pharmaceutical compositions is in the form of different drug delivery systems such as drug loaded microparticles, molded implants, coated granules, injectable sustained release particles, stents, films, matrix tablet, coated tablets, dry syrup, mouth dissolving tablets, microparticles dispersed in gels, taste masked formulation, inserts (ophthalmic), fibers, ligatures and sutures.

### Related Applications

This application claims priority from India National patent application serial No. 355/MUM/2003, filed 10 April 2003.

### TECHNICAL FIELD

The present invention relates to novel biodegradable aliphatic polyesters derived from fatty diacids and fatty diols with even number of carbon atoms, pharmaceutical compositions and applications thereof. More particularly pharmaceutical composition comprises at least one pharmaceutically active ingredient and the said biodegradable aliphatic polyester in the form of various drug delivery systems.

### BACKGROUND AND PRIOR ART

With the increasing demand for biodegradable/biocompatible polymers, aliphatic polymers like poly-lactic acid, poly-glycolide, copolymers of lactide-glycolide are gaining importance. These polymers are synthesized mostly by either ring opening polymerization or by direct polycondensation technique. These polyesters are very expensive as compared to the other polymers, which are widely used. Because of its very high cost, these polyesters find very less commercial usage but because of the GRAS status, these polymers are in great demand. In the pharmaceutical field, the trend in drug delivery has been towards biodegradable polymer excipients for controlled release formulation and for implants as it would not require follow-up surgical removal once the drug supply is depleted. The most widely investigated and advanced polymer with regard to available toxicological and clinical data, are the aliphatic polyesters based on glycolide/lactide, which include poly-lactic acid, poly-glycolide, copolymers of lactide-glycolide. Various applications of these polymers are reported in literature, for example U.S. Pat. No. 5,478,564 and U.S. Pat. No. 5,609,886 describe a preparation method for the microparticles of copolymers of lactic acid and glycolic acid for controlled release of water soluble pharmaceutically active agents.

U.S. Pat. No. 5,705,197 describes methods for preparation of microparticles of vaccine and biologically active substances using poly-(D, L)lactide co-glycolide.

U.S. Pat. No. 5,718,922 discusses the use of microparticles of copolymers of lactic acid and glycolic acid for intravitreal injection containing antiviral agents for CMV retinitis.

U.S. Pat. No. 6,159,502 discusses microparticles of poly (lactic acid) and its copolymers coupled with a carrier for oral delivery of substances to the circulation or lymphatic drainage of the host. The carriers are mucosal binding proteins, bacterial adhesions, viral adhesions, toxic binding subunits, lectins, Vitamin B.sub.12 and analogues or derivatives of Vitamin B.sub.12 possessing binding activity to Castle's intrinsic factor.

U.S. Pat. No. 6,296,667 uses poly-lactic acid as a bone substitute and U.S. Pat. No. 6,338,859 uses poly-lactic acid, poly-glycolic acid, poly-(D-lactic acid), poly-(D, L-lactic acid), lactide/glycolide copolymers for micelle formation along with poly-vinyl pyrrolidone for the delivery of anti cancer drugs.

U.S. Pat. No. 6,511,748 mentions the use of PLA and PGA as core material in bioabsorbable fibers, which are used, for fracture fixation and spinal fusion

Poly-(lactide/glycolide) has been used to make films containing antibiotics for the insertion into the periodontal pocket as described in Journal of Controlled Release 3(1993), 137-146.

Various methods are described in literature for the synthesis of biodegradable polymers other than PLA, PLGA.

U.S. Pat. No. 6,515,054 and related patents use filler along with the biodegradable polymer to lower the cost of the polymer and to accelerate biodegradation

U.S. Pat. No. 6,303,677 describes a method for preparing a method biodegradable polymer by using adipic acid or ester forming derivatives or terphthalic acid and C-2 to C-6 substituted alkanediols or C-10 cycloalkanediols. These polymers have been used for making moldings or are blended with starch to obtain moulds.

U.S. Pat. No. 6,201,072 highlights a biodegradable ABA or BAB type of triblock polymer of poly-(lactide-co-glycolide) and polyethylene glycol which possesses reverse thermal gelation properties. This polymer has been used for pharmaceutical applications.

U.S. Pat. No. 6,133,404 describes a biodegradable polymer containing at least one aliphatic dicarboxylic acid containing 2-14 carbon atoms and at least one aromatic or alicyclic carboxylic acid and one glycol. This polymer exhibits good mechanical strength and can replace pre-existing expensive aliphatic polyesters. No pharmaceutical use of this polymer is reported

Patent Application WO 0055236 describes a method for synthesizing aliphatic polyesters using aliphatic dicarboxylic acids and aliphatic glycols. This polymer finds applications in various fields like food packing material, films, semi-expanded and expanded products, fibers, fabrics, composites with mineral and vegetable filler, bottles for food, cosmetics and pharmaceutical field

U.S. Pat. No. 5,919,835 describes polymer blends of two or more polyanhydrides and polyester or their mixtures as a carrier for pharmaceutically active agent

U.S. Pat. No. 5,585,460 highlights a method for synthesis of high molecular weight aliphatic polyester of lactic acid and glycolic acid to obtain microparticles of the polyester containing the medicament for pharmaceutical application.

As described above PLA, PLGA polyesters have a wide range of pharmaceutical applications. However this class of polyester has two main disadvantages, limited hydrolytic stability because of high concentration of ester linkages on the backbone, which leads to their hydrolysis in the presence of atmospheric moisture and high cost which is a limiting factor on its commercial usage.

Hence it is essential to have polyesters, which are less expensive, hydrolytically stable, have good mechanical properties, are easy to synthesize, biodegradable, biocompatible and safe for use in living organisms.

It is known that fats undergo metabolism in the liver by beta-oxidation where two carbon atoms in the fatty acid chain are removed in each cycle and hence fatty diacids with even number of carbon atoms are easily metabolized. Fatty diacids having odd number of carbon atoms are toxic due to formation of formic acid as a degradation product.

Polyesters of diols containing even number of carbon atoms and diacids with even number of carbon atoms could be synthesized by conventional condensation polymerization technique, direct polymerization technique.

Other patents describe the use of diacids and diols for aliphatic polyester synthesis for engineering applications. However the toxicity and in-vitro biodegradation of these polymers is not extensively studied. Also the use of these polymers for pharmaceutical applications is not completely described.

In the present invention, synthesis of polyesters of diols and diacids which contain even number of carbon atoms, which are biodegradable and non-toxic to living animals and could be used for a wide pharmaceutical application at a much lower cost are described.

Objective

An object of the present invention is to develop a pharmaceutical composition using aliphatic polyester which has good stability, excellent mechanical properties, is easy to synthesize, less expensive, biodegradable, biocompatible and safe for use in living animals in the form of different drug delivery system.

### SUMMARY OF THE INVENTION

Novel biodegradable aliphatic polyesters derived from fatty diacids and fatty diols with even number of carbon atoms, pharmaceutical compositions and applications thereof wherein the said pharmaceutical compositions comprises at least one pharmaceutically active ingredient and the said biodegradable aliphatic polyester and the said pharmaceutical compositions are in the form of different drug delivery systems such as drug loaded microparticles, molded implants, coated granules, injectable sustained release particles, stents, films, matrix tablet, coated tablets, dry syrup, mouth dissolving tablets, microparticles dispersed in gels, taste masked formulation, inserts (ophthalmic), fibers, ligatures and sutures.

A biodegradable aliphatic polymer is synthesized by conventional condensation polymerization method from a diol and diacid as the starting material, both of which contain even number of carbon atoms in the presence of a catalyst. Para-toluene sulphonic acid is used as the catalyst. Solid-state condensation is carried out to increase the molecular weight.

The polymer thus formed can be used for making various pharmaceutical formulations which include drug loaded microparticles by suitable process, molded implants, coated granules, injectable sustained release particles, stents, films, matrix tablets, coated tablets, dry syrup, mouth dissolving tablets, microparticles dispersed in gels, taste masked formulation, inserts, fibers, ligatures and sutures.

### DETAILED DESCRIPTION

The present invention is related to development of various pharmaceutical compositions using biodegradable aliphatic polyesters synthesized by conventional condensation polymerization technique from diols containing 2-20 carbon atoms and dicarboxylic acid containing 1-50 carbon atoms. The number of carbon atoms for diols and carboxylic acid is not a limiting factor but both containing even number of carbon atoms and terminal carboxy groups is essential. The synthesis is carried out in two steps by using two different catalyst which are, para-toluene sulphonic acid in esterification step and Zinc acetate in condensation step. Carbon-dioxide is treated as a dicarboxylic acid as a special case and can be incorporated by transesterification using dimethylcarbonate or ethylene carbonate.

Aliphatic polyesters of various molecular weights could be obtained by this method by varying conditions in the condensation step.

Aliphatic polyester synthesized by this method has good thermal stability and excellent mechanical properties. The in-vitro degradation of the polymer occurs in the presence of lipase to low molecular weight compounds. The synthesized class of polyester would undergo same degradation pattern in living animals.

On determination as per OECD guidelines, the LD₅₀ of the synthesized polymer is observed to be more than 2000 mg/kg of body weight when tested in male albino mice. When said polymer is administered for a prolonged period of time, no tissue accumulation is seen in mice indicating its biodegradability in living animals. Hence this polymer could be used safely in all living animals.

The biodegradability and safe toxic limits of these aliphatic polyesters makes them useful in pharmaceutical applications.

The biodegradable aliphatic polyester obtained by this invention is used as the base for microcapsules. The sustained release microcapsules containing a water insoluble drug can be produced by preparing an oil/water suspension system, in which the medicament is embedded within the polymer particles, which forms the oil phase, and the aqueous phase contains stabilizing agents for the microparticles. The stabilizing agent forms a thin protective layer around the droplets and hence reduces the extent of droplet coalescence and coagulation. The stabilizing agents, which could be used, are polyvinyl alcohol, polyvinyl pyrrolidone, alginate, gelatin, methyl cellulose, polyoxyethylene derivatives of sorbitan fatty esters [Tweens] and polyoxyethylene fatty ethers [Brij].

The micro/nano particles are prepared by either solvent-evaporation technique or solvent extraction technique. The micro/nano particles thus formed by this method are made into various dosage forms for administration by the living animals. The dosage forms are tablets, sustained release granules filled in capsules, microparticulate implants for periodontitis, microparticulate implants for synovial joint and other such formulations. The polymer is used for coating the granules in order to get sustained action and for preparation of biodegradable stents.

The particle size of the nano/micro particles derived from the novel biodegradable aliphatic polyester is in the range of 10 nm to 1000 microns depending on the type and concentration of stabilizer and drug to polymer ratio used in the formulation.

The drug to polymer ratio in pharmaceutical compositions is selected from 95:5 to 1:99.

In addition to the microparticles, the polymer could be molded into different shapes by melting the polymer and dispersing the medicament to obtain implants for the sustained release of the medicament for prolonged period of time. The polymer implant could be in circular, cylindrical or any other molded form.

A biodegradable aliphatic polyester derived from fatty diacids and fatty diols with even number of carbon atoms, a pharmaceutical compositions comprises at least one pharmaceutically active ingredient and the said biodegradable aliphatic polyester in the form of drug delivery systems like drug loaded microparticles by suitable process, molded implants, coated granules; injectable sustained release particles, stents, films matrix tablets, coated tablets, drug syrup, mouth dissolving tablets, microparticles dispersed in gels, taste masked formulation, inserts, fibers, ligatures and sutures.

The drug delivery system of novel biodegradable aliphatic polyesters derived from fatty diacids and fatty diols with even number of carbon atoms is drug-loaded micro/nano particles.

The drug delivery system of novel biodegradable aliphatic polyesters derived from the fatty diacids and fatty diols with even number of carbon atoms are molded implants containing drug.

The drug delivery system of novel biodegradable aliphatic polyesters derived from the fatty diacids and fatty diols with even number of carbon atoms are coated granules, prepared by coating the granules with 1-5% solution of the said biodegradable aliphatic polyester in a suitable solvent.

The drug delivery system of novel biodegradable aliphatic polyesters derived from the fatty diacids and fatty diols with even number of carbon atoms are injectable sustained release microparticles suitable for sub-cutaneous, intra-muscular or periodontal administration for sustained action for the required period.

The drug delivery system of novel biodegradable aliphatic polyesters derived from the fatty diacids and fatty diols with even number of carbon atoms in the form of stents is prepared by molding the said biodegradable aliphatic polyester into stents after being ablated with laser.

The drug delivery system of novel biodegradable aliphatic polyesters derived from the fatty diacids and fatty diols with even number of carbon atoms in the form of microparticles dispersed in gel is prepared by incorporating the micro particles in a gel suitable for use in the treatment of periodontitis.

The drug delivery system of novel biodegradable aliphatic polyesters derived from the fatty diacids and fatty diols with even number of carbon atoms in the form of films is self supporting drug loaded films.

The number average molecular weight of biodegradable aliphatic polyester in the present invention is not especially limited, but is, for example, in the range of 3,000 to 30,000.

Preferred molecular weight of biodegradable aliphatic polyester used to prepare drug delivery system in the form of coated granules is in the range 3,000 to 7,000.

Preferred molecular weight of biodegradable aliphatic polyester used to prepare taste-masked formulation is in the range of 3,000 to 7,000.

Preferred molecular weight of biodegradable aliphatic polyester for drug delivery systems in the form of matrix or coated tablets by melt granulation is in the range of 3,000 to 7,000.

Preferable molecular weight of biodegradable aliphatic polyester used to prepare drug delivery system in the form of molded implants and films are in the range of 7,000 to 30,000.

Preferred molecular weight of biodegradable aliphatic polyester for drug delivery system, stents is 30,000 onwards.

Pharmaceutical compositions mentioned above are prepared with or without lipase.

The class of drugs which could be used are anti-hypertensives, cardiovascular agent analgesics, steroids, physiologically active peptides and/or proteins, anti-cancer agents, antibiotics, fibrinolytics, anti-inflammatory agents, expectorants, muscle relaxants, epilepsy remedies, anti-ulcerative agents, anti-hyperchondriac agents, anti-allergic agents, diuretics diabetes curatives, hyperlipidemic remedies, anticoagulants, hemolytic agents, anti tubercular agents, hormones, anesthetic antagonists, osteoclastic suppressants, osteogenic promotives, angiogenesis suppressors, mydriatics, myotics,glaucoma therapy and or mixtures thereof.

Advantages
The said aliphatic polyesters are easy to synthesize and inexpensive to manufacture on a commercial scale.
The synthesized polymers are readily biodegradable and do not show any toxic effect as these are metabolized by normal lipid metabolism in the liver of living animals.
The said polymer can be easily made into various dosage forms of wide pharmaceutical applications containing several classes of pharmaceutically active agent.
The said polymer is stable at room temperature and does not need any specific storage/working conditions.

### EXAMPLES

The invention is illustrated by way of examples as follows,

### Example 1

A 500 ml three-necked flask equipped with a stirrer and condenser is charged with ethylene glycol and sebacic acid in a molar ratio of 2:1. 0.1% para-toluene sulphonic acid is added as the catalyst. The temperature is gradually increased up to 130° C. with vigorous stirring. The reaction is continued until the distillation of water is completed. 1% zinc acetate is added to carry out the condensation reaction for building up the molecular weight. The reaction is carried out under high vacuum and at a temperature of 180° C. The polyester with the desired molecular weight is formed after 300 minutes of condensation reaction. Solvent evaporation method is used to prepare microparticles in which pharmaceutically active agent [drug] to be encapsulated is added to 5% solution of polymer in dichloromethane. This solution is added to a solution of stabilizer with stirring at 2000 rpm. Stirring is continued for one hour at room temperature to evaporate dichloromethane. The microparticles formed are collected by centrifugation and are dried in vacuum to give a dry powder.

### Example 2

The polymer described in example 1 is used for the formulation of microparticles using 1% PVA as the stabilizing agent. The drug:polymer ratio is varied from 1:5 to 1:1. The solvent to non-solvent ratio is fixed at 1:25. The drug entrapment for a water insoluble drug is found to be in the range of 55-90% and that for a water soluble drug is found to be 2-12%

### Example 3

The polymer described in example 1 is used for the formulation of microparticles using 0.5% PVA as the stabilizing agent. The drug:polymer ratio is varied from 1:5 to 1:1. The solvent to non-solvent ratio is fixed at 1:25. The drug entrapment for a water insoluble drug is found to be in the range of 70-90% and that for a water soluble drug is found to be 2-12%

### Example 4

The polymer described in example 1 is used for the formulation of microparticles using 0.25% PVA as the stabilizing agent. The drug:polymer ratio is varied from 1:5 to 1:1. The solvent to non-solvent ratio is fixed at 1:25. The drug entrapment for a water insoluble drug is found to be in the range of 80-90% and that for a water soluble drug is found to be 2-12%

### Example 5

The polymer described in example 1 is used for the formulation of microparticles using 1% Pluronic F68 as the stabilizing agent. The drug:polymer ratio is varied from 1:5 to 1:1. The solvent to non-solvent ratio is fixed at 1:25. The drug entrapment for a water insoluble drug is found to be in the range of 60-80% and that for a water soluble drug is found to be 2-12%

### Example 6

The polymer described in example 1 is used for the formulation of microparticles using 0.5% Pluronic F68 as the stabilizing agent. The drug:polymer ratio is varied from 1:5 to 1:1. The solvent to non-solvent ratio is fixed at 1:25. The drug entrapment for a water insoluble drug is found to be in the range of 60-90% and that for a water-soluble drug is found to be 2-12%

### Example 7

The polymer described in example 1 is used for the formulation of microparticles without using stabilizing agent. The drug:polymer ratio is varied from 1:5 to 1:1. The solvent to non-solvent ratio is fixed at 1:25. The drug entrapment for a water insoluble drug is found to be in the range of 60-90% and that for a water-soluble drug is found to be 2-12%

### Example 8

The microparticles formulated in examples 2-7 are analyzed for particle size. The mean particle size is found to be in the range of 5-30 microns, depending on the type and concentration of stabilizer used and the drug:polymer ratio.

### Example 9

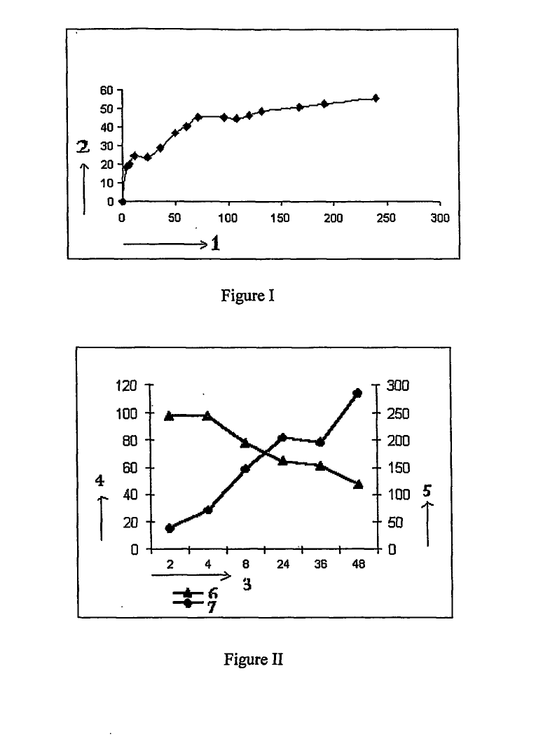

The release profile of the formulation described in example 7 is shown in FIG. 1 Release profile of formulation where

1] Drug: Rofecoxib

2] Drug:polymer ratio is 1:5

3] solvent:non-solvent ratio is 1:25

### Example 10

The polymer synthesized in example 1 is subjected to degradation by lipase. Degradation of the polymer with lipase is shown in FIG. 2. Molecular weight of the polymer=4195

### Example 11

Moulded Implant

The polymer is melted and the pharmaceutically active agent is dispersed in the melted polymer, which is then poured into moulds to form implants of desired size and shape.

### Example 12

Coated Granules

A 1-5% solution of polymer is made in a suitable solvent This solution is applied to the granules containing the pharmaceutically active agent to be coated in a coater. The extent of coating is dependent on the final use of the granules.

### Example 13

Microparticales Dispersed in Gel

The microparticles formed in examples 2-7 are incorporated in a gel suitable for use in the treatment of periodontitis.

### Example 14

Injectable Sustained Release Particles

The microparticles formed in examples 2-7 are suitable for sub-cutaneously or intra-muscularly administration for sustained action for the required period of time.

### Example 15

Stents

The aliphatic polyester is molded into stents after being ablated with laser.

### Example 16

Controlled Release Intra-Synovial Formulation

The microparticles formed in examples 2-7 are suitable for controlled release intra-synovial formulation.

### Example 17

Films

The aliphatic polyester can form self supporting drug loaded films.

### Example 18

Taste Mask Formulation

The aliphatic polyester can be used for taste masking.

Lipase is incorporated into the microcapsules, implants, films to modify the release of the drug.

### DESCRIPTION OF DRAWING

Figure I illustrates dissolution or release profile of the formulation described in the example 7. Number 1 indicates time in hours and 2 indicates % drug release.

Figure II illustrates time dependent polymer degradation when biodegradable aliphatic polyester subjected to degradation by lipase. Number 3 indicates time in hours, 4 indicates % decrease in molecular weight and 5 indicates acid value. Number 6 indicates polymer degradation pattern with respect to % decrease in molecular weight. Polymer degradation pattern with respect to acid value is shown by number 7.

While the present invention is described above in connection with preferred or illustrative embodiments, these embodiments are not intended to be exhaustive or limiting of the invention. Rather, the invention is intended to cover all alternatives, modifications and equivalents included within its scope, as defined by appended claims.

We claim:

1. Novel biodegradable aliphatic polyesters derived from fatty diacids and fatty diols both with even number of carbon atoms, in which the even carbon number is selected from 2-50, pharmaceutical compositions and applications thereof wherein the said pharmaceutical compositions comprises at least one pharmaceutically active ingredient and the said biodegradable aliphatic polyester derived from fatty diacids and fatty diols both with even number of carbon atoms such as 2-50;
wherein the said pharmaceutical compositions are in the form of different drug delivery systems such as drug loaded microparticles, nanoparticles, molded implants, coated granules, injectable sustained release particles, stents, films, matrix tablet, coated tablets, dry syrup, mouth dissolving tablets, microparticles dispersed in gels, taste masked formulation, inserts (ophthalmic), fibers, ligatures and sutures.

2. Novel biodegradable non-toxic aliphatic polyesters derived from the fatty diacids and fatty diols as claimed in claim 1 wherein the said fatty diacids with one carbon atom, particularly, Carbon di-oxide as carbonic acid, H₂CO₃, may also be used to prepare the said biodegradable aliphatic polyester.

3. Novel biodegradable non-toxic aliphatic polyesters derived from the fatty diacids and fatty diols with even number of carbon atoms as claimed in claim 1 to 2 wherein the said biodegradable aliphatic polyesters derived from the fatty diacids and fatty diols in the molar ratio of 1:1 or could vary from 0.97:1 to 1:1.03 depending on the end group required.

4. Novel biodegradable aliphatic polyesters derived from the fatty diacids and fatty diols with even number of carbon atoms as claimed in claims 1 to 3 wherein molecular weight of the said biodegradable aliphatic polyesters is in the range of 3,000 to 30,000.

5. Novel biodegradable aliphatic polyesters derived from the fatty diacids and fatty diols with even number of carbon atoms as claimed in claims 1 to 4 wherein LD₅₀ of the said biodegradable aliphatic polyesters is more than 2000 mg/Kg of body weight of mice.

6. Novel biodegradable aliphatic polyesters derived from the fatty diacids and fatty diols with even number of carbon atoms as claimed in claims 1 to 5 wherein the said biodegradable aliphatic polyesters has thermal stability and excellent mechanical properties.

7. Novel biodegradable aliphatic polyesters derived from the fatty diacids and fatty diols with even number of carbon atoms as claimed in claims 1 to 6 wherein the said pharmaceutically active ingredient is selected from anti-hypertensives, cardiovascular agents, analgesics, steroids, physiologically active peptides and/or proteins, anti-cancer agents, antibiotics, fibrinolytics, anti-inflammatory agents, expectorants, muscle relaxants, epilepsy remedies, anti-ulcerative agents, anti-hyperchondriac agents, anti-allergic agents, diuretics diabetes curatives, hyperlipidemic remedies, anticoagulants, hemolytic agents, anti tubercular agents, hormones, anesthetic antagonists, osteoclastic suppressants, osteogenic promotives, angiogenesis suppressors, mydriatics, myotics, glaucoma therapy and or mixtures thereof.

8. The drug delivery system of novel biodegradable aliphatic polyesters derived from fatty diacids and fatty diols with even number of carbon atoms as claimed in claims 1 to 7 wherein the said drug delivery system is drug-loaded micro/nano particles.

9. The drug delivery system of novel biodegradable aliphatic polyesters derived from the fatty diacids and fatty diols with even number of carbon atoms as claimed in claims 1 to 7, wherein the said drug delivery systems are molded implants containing drug.

10. The drug delivery system of novel biodegradable aliphatic polyesters derived from the fatty diacids and fatty diols with even number of carbon atoms as claimed in claims 1 to 7 wherein the said drug delivery systems are coated granules, prepared by coating the granules with 1-5% solution of the said biodegradable aliphatic polyester in a suitable solvent.

11. The drug delivery system of novel biodegradable aliphatic polyesters derived from the fatty diacids and fatty diols with even number of carbon atoms as claimed in claims 1 to 7 wherein the said drug delivery systems are injectable sustained release microparticles suitable for sub-cutaneous, intramuscular or periodontal administration for sustained action for the required period.

12. The drug delivery system of novel biodegradable aliphatic polyesters derived from the fatty diacids and fatty diols with even number of carbon atoms in the form of stents as claimed in claims 1 to 7 wherein the said stent form is prepared by molding the said biodegradable aliphatic polyester into stents after being ablated with laser.

13. The drug delivery system of novel biodegradable aliphatic polyesters derived from the fatty diacids and fatty diols with even number of carbon atoms in the form of microparticles dispersed in gel as claimed in claims 1 to 7 wherein the said drug delivery system in gel form is prepared by incorporating the micro particles in a gel suitable for use in the treatment of periodontitis.

14. The drug delivery system of novel biodegradable aliphatic polyesters derived from the fatty diacids and fatty diols with even number of carbon atoms in the form of films as claimed in claims 1 to 7 wherein the said drug delivery system in the form of film is self supporting drug loaded films.

15. Novel biodegradable aliphatic polyesters derived from fatty diacids and fatty diols with even number of carbon atoms, pharmaceutical compositions and applications thereof as claimed in claims 1 to 8, 11 and 13 wherein the stabilizing agents are selected from polyvinyl alcohol, polyvinyl pyrrolidone, alginate, gelatin, methyl cellulose, polyoxyethylene derivatives of sorbitan fatty esters and polyoxyethylene fatty ethers.

16. Novel biodegradable aliphatic polyesters derived from fatty diacids and fatty diols with even number of carbon atoms, pharmaceutical compositions and applications thereof as claimed in claim 1 to 15 wherein the drug to polymer ratio is selected from 95:5 to 1:99.

17. Novel biodegradable aliphatic polyesters derived from fatty diacids and fatty diols with even number of carbon atoms, pharmaceutical compositions and applications thereof as claimed in claim 1 to 8, 11 and 13 wherein particle size of microparticles is in the range of 10 nm to 1000 microns depending on the type and concentration of stabilizer and drug to polymer ratio used in the formulation.

18. The drug delivery system of novel biodegradable aliphatic polyesters derived from the fatty diacids and fatty diols with even number of carbon atoms in the form of drug loaded microparticles, nanoparticles, molded implants, coated granules, injectable sustained release particles, stents, films, matrix tablet, coated tablets, dry syrup, mouth dissolving tablets, microparticles dispersed in gels, inserts (ophthalmic), fibers, ligatures and sutures as claimed in claims 1 to 17 wherein the said drug delivery systems are with or without the addition of lipase to modify the drug release.

19. Novel biodegradable aliphatic polyesters derived from fatty diacids and fatty diols with even number of carbon atoms, pharmaceutical compositions and applications thereof as claimed in claims 1 to 18 wherein the said pharmaceutical compositions could be administered by either oral, ophthalmic, parenteral, mucosal or transdermal route.

20. Novel biodegradable aliphatic polyesters derived from fatty diacids and fatty diols with even number of carbon atoms, pharmaceutical compositions and applications thereof as substantially described herein with reference to foregoing examples 1 to 18.
